(19) United States
(12) Patent Application Publication
(10) Pub. No.: US 2009/0280108 A1
(43) Pub. Date: Nov. 12, 2009

(54) SERUM AMYLOID A PROTEIN IN INFLAMMATION AND OBESITY

(76) Inventors: Da-Wei Gong, Olney, MD (US); Alan R. Shuldiner, Columbia, MD (US); Rong-Ze Yang, Baltimore, MD (US); Susan K. Fried, Baltimore, MD (US)

Correspondence Address: MERCHANT & GOULD PC, P.O. BOX 2903, MINNEAPOLIS, MN, 55402-0903, US

(21) Appl. No.: 11/721,363

(22) PCT Filed: Dec. 9, 2005
(86) PCT No.: PCT/US05/44578

§ 371 Date: Sep. 20, 2007

Related U.S. Application Data
(60) Provisional application No. 60/634,816, filed on Dec. 10, 2004.

(51) Int. Cl. A61K 39/395; C12Q 1/02

(52) U.S. Cl. 424/130.1; 435/29; 435/6

(57) ABSTRACT

The present invention relates to the discovery that acute-phase serum amyloid protein A (A-SAA) is a biomarker for obesity and certain abnormal conditions. The present invention, therefore, provides methods of diagnosing obesity or an abnormal condition in a subject The present invention also provides methods of monitoring the progression of obesity or an abnormal condition in a subject. The present invention also relates to treating obesity or an abnormal condition comprising reducing the levels of active SAA1 and/or SAA2 in a subject in need thereof.

### STATEMENT REGARDING FEDERALLY SPONSORED RESEARCH OR DEVELOPMENT

Part of the work performed during development of this invention utilized U.S. Government funds awarded by National Institutes of Health, Grant Nos. HL/DK62093 and HL/DK57835. The U.S. Government has certain rights in this invention.

### BACKGROUND OF THE INVENTION

1. Field of the Invention

The present invention relates to the discovery that acute-phase serum amyloid protein A (A-SAA) is a biomarker for obesity and certain abnormal conditions. The present invention, therefore provides methods of diagnosing obesity or an abnormal condition in a subject comprising measuring levels of serum amyloid A protein 1 (SAA1) and/or serum amyloid A protein 2 (SAA2) and comparing these measured levels with accepted normal levels of SAA1 and/or SAA2, respectively, where a difference between the subject's levels and normal levels are indicative of obesity or the presence of an abnormal condition in the subject. The present invention also provides methods of monitoring the progression of obesity or an abnormal condition in a subject, comprising measuring levels of serum amyloid A protein 1 (SAA1) and/or serum amyloid A protein 2 (SAA2) at two different time points in a subject, and comparing these measured levels at the two time points to determine a difference in levels of SAA1 and/or SAA2 over time, respectively, where a difference over time is indicative of the progression or regression of obesity or an abnormal condition in the patient. The present invention also relates to treating obesity or an abnormal condition comprising reducing the levels of active SAA1 and/or SAA2 in a patient in need thereof.

2. Background of the Invention

Currently, over 100 million Americans are categorized as considered overweight or obese, and obesity causes at least 300,000 deaths annually in the United States alone. Obesity is considered a chronic disease with a strong genetic component. In addition, obesity can increase the risk of developing conditions such as, but not limited to, high blood pressure, type-2 diabetes, heart disease, stroke, gallbladder disease and some forms of cancer.

There is increasing evidence that a low-grade chronic inflammation is associated with obesity an that this state of chronic inflammation may be an important mediator of metabolic syndromes which include obesity, type-2 diabetes, dyslipidemia, and atherosclerosis, to name a few (Xu H, et al., J Clin Invest 112:1821-1830 (2003); Weisberg S P, et al., J Clin Invest 112:1796-1808 (2003); Lehrke M, et al., Nat Med 10:126-127 (2004); Reaven P, J Insur Med 36:132-142 (2004); Yudkin J S, Int J Obes Relat Metab Disord 27 Suppl 3:S25-28 (2003); Schmidt M I, et al., Clin Chem Lab Med 41:1120-1130 (2003), all of which are incorporated by reference). Indeed, the increased mass of dysfunctional adipose tissue in obesity is a source of several of factors that have traditionally been regarded as pro-inflammatory factors, including tumor necrosis factor alpha (TNFα), interleukin-6 (IL-6) and monocyte chemotactic protein-1 (MCP-1), as well as the pro-thrombotic factor plasminogen activator inhibitor-1 (PAI-1).

On the other hand, acute phase proteins such as C-reactive protein (CRP), alpha-1 antitrypsin, alpha 1-antichymotrypsin, alpha 1-antimacroglobulin, fibrinogen, prothrombin, factor VIII, von Willebrand factor, plasminogen and serum amyloid A protein (SAA) increase dramatically in serum in response to acute inflammation and trauma, whereas chronic inflammation usually exhibits only modest increases in circulating levels of many of these acute phase reactants. In population studies, however, circulating levels of some of these acute phase reactants are predictive of cardiovascular risk.

Of the acute phase protein, SAA is multigene protein family that is actually associated with both chronic and acute inflammation and consists of four genes (SAA1-4) that are conserved in major vertebrates, see Sellar G C, et al., Genomics 19:221-227 (1994). In humans, only three of the four genes (SAA1, 2 and 4), are expressed, see Kluve-Beckerman B, et al., DNA Cell Biol 10:651-661 (1991). In response to acute inflammatory stimuli, plasma levels of SAA1 and SAA2 may increase as much as about 1000-fold in a period of about 5-6 hours, (Cabana V G, et al., J Lipid Res 30:39-49 (1989)), whereas SAA4 is marginally regulated (Whitehead A S, et al., J Biol Chem 267:3862-3867 (1992)). Accordingly, SAA1 and SAA2 are collectively referred to as acute-phase SAA (A-SAA), and SAA4 is referred to as constitutive SAA (C-SAA).

SAA is involved in inflammation and is induced by numerous proinflammatory stimuli such as lipopolysaccharide (LPS), tumor necrosis factor-α (TNFα), interleukin (IL)-1, IL-6 and IL-8, see Hagihara K, et al., Biochem Biophys Res Commun 314:363-369 (2004); Bopst M, et al., Eur J Immunol 28:4130-4137 (1998). Moreover, SAA is a potent stimulus for the gene expression or release of TNFα, IL-6 and IL-8 in neutrophils, see Furlaneto C J, et al., Biochem Biophys Res Commun 268:405-408 (2000); Hatanaka E, et al., Immunol Lett 91:33-37 (2004); Ribeiro F P, et al., Mediators Inflamm 12:173-178 (2003).

Thus, the invention provides methods and compositions that focus of levels of SAA1 and/or SAA2 for diagnosing, monitoring and treating obese subjects and abnormal conditions associated therewith.

### SUMMARY OF THE INVENTION

The present invention relates to the discovery that acute-phase serum amyloid protein A (A-SAA) is a biomarker for obesity and certain abnormal conditions. The present invention, therefore provides methods of diagnosing obesity in a subject comprising measuring levels of serum amyloid A protein 1 (SAA1) and/or serum amyloid A protein 2 (SAA2) and comparing these measured levels with accepted normal levels of SAA1 and/or SAA2, respectively, where a difference between the subject's levels and normal levels are indicative of obesity in the subject.

The present invention also provides methods of diagnosing an abnormal condition in a subject comprising measuring levels of serum amyloid A protein 1 (SAA1) and/or serum amyloid A protein 2 (SAA2) and comparing these measured levels with accepted normal levels of SAA1 and/or SAA2, respectively, where a difference between the subject's levels and normal levels are indicative of the presence of an abnormal condition in the subject.

The present invention also provides methods of monitoring the progression of obesity or abnormal condition in a subject, comprising measuring levels of serum amyloid A protein 1 (SAA1) and/or serum amyloid A protein 2 (SAA2) at two different time points in a subject, and comparing these measured levels at the two time points to determine a difference in levels of SAA1 and/or SAA2 over time, respectively, where a difference over time is indicative of the progression or regression of obesity or an abnormal condition in the patient.

The present invention also relates to treating obesity and/or abnormal conditions comprising reducing the levels of active SAA1 and/or SAA2 in a patient in need thereof.

The present invention also relates to compositions, such as antibodies, that can be used in the various diagnosis and treatment methods presented herein.

### BRIEF DESCRIPTION OF THE DRAWINGS

FIG. 1 depicts the tissue-restricted expression of A-SAA mRNA. FIG. 1A represents an RT-PCR analysis of SAA and β-actin gene expression in stromal vascular cells (SVCs) and fat cells (FCs) fractionated from human omental (O) and subcutaneous (S) fat tissues. FIGS. 1B and 1C represent Northern analyses of multiple tissue blots from the human and mouse, respectively. For all Northern analyses, about 15 μg of total RNAs from indicated tissues were blotted onto a nylon membrane and hybridized with a radiolabeled human (FIG. 1B) or murine (FIG. 1C) SAA2 cDNA probe. The RNA loadings were visualized by ethidium bromide staining.

FIG. 2 depicts acute-SAA being positively correlated with body mass index (BMI.) SAA levels were measured in plasma of normal human subjects who were divided into lean (BMI<25 kg/m², n=54), overweight (BMI 25-30 kg/m², n=49) and obese (BMI≧30 kg/m², n=31) groups. Data are expressed as mean±SEM (ln-transformed for analysis, back-transformed for presentation), adjusted for age, sex and family structure. * p=0.013 vs. lean group.

FIG. 3 depicts decreases in serum A-SAA is with weight loss by diet and exercise. FIG. 3A shows serum A-SAA levels and BMI before and after weight loss through diet and exercise. Data are expressed as mean±SEM, n=24. Paired t-test was performed after log-transformation. ** p<0.01. FIG. 3B represents the correlation between changes of serum A-SAA level and changes in body fat mass, pre- and post-weight loss. R=0.55, p<0.01, n=24.

FIG. 4 depicts the effect of rosiglitazone on serum A-SAA levels and adipose A-SAA production in humans. Serum A-SAA (n=8) and adipose secretion (n=7) of A-SAA ex vivo were measured in non-diabetic human subjects before and after 3 months of rosiglitazone treatment. The data are plotted with lines connecting the level of each individual. Serum A-SAA and adipose secretion of SAA from the individuals (identical symbols in each panel represent the same individual for both studies) were significantly decreased by rosiglitazone (paired t-test after log-transformation (P<0.01 for both comparisons)).

FIG. 5 depicts the actions of rosiglitazone by directly suppressing A-SAA production in adipose tissue in vitro. Adipose tissues were incubated in cell culture medium 199 (basal) or medium with insulin (Ins, 7 nM) and dexamethasone (Dex, 25 nM) in the presence or absence of rosiglitazone (Rosi, 1 μM for about 48 hours. SAA production between 24 to 48 hours was measured and corrected for tissue weight. Data are expressed as mean±SEM, n=3. **P<0.01, unpaired t-test after log-transformation.

FIG. 6 depicts the effects of A-SAA as a mediator of inflammatory cytokines. Human coronary vascular endothelial cells (HCVECs) (FIG. 6A) and mouse monocytes RAW264 (FIG. 6B) were treated with vehicle (phosphate-buffered saline, white bar), low (0.47 μg/ml, hatched bar) or high (2.34 μg/ml, black bar) concentrations of SAA for about 8 hours in serum-free medium. Cell-free supernatants were assayed for cytokines by Luminex. Data are expressed as mean±SEM from 3-4 independent experiments. Statistical significance (*p<0.05; **P<0.01, unpaired t-test) was observed between the SAA-treated group and control.

### DETAILED DESCRIPTION OF THE INVENTION

Serum amyloid A (SAA) is a member of acute phase proteins, whose serum level rises dramatically in response to acute inflammation and trauma. The liver has been considered the primary source of SAA production, although extra-hepatic expression has also been reported. In this application, the inventors disclose and teach that in humans, acute-phase SAA (A-SAA), i.e., SAA1 and SAA2, is predominantly expressed in adipose tissue. This adipose-specific expression in humans is in sharp contrast to mice where A-SAA is expressed predominantly in liver. The high relative abundance of A-SAA mRNA in human adipose tissue, as described herein, provides further support to the evolving concept that adipose tissue is an important source of inflammatory factors, which have important effects on local fat metabolism as well as systemic effects in liver, muscle, cells of the immune system, and the vasculature.

The present invention, therefore provides methods of diagnosing obesity in a subject comprising measuring levels of serum amyloid A protein 1 (SAA1) and/or serum amyloid A protein 2 (SAA2) and comparing these measured levels with accepted normal levels of SAA1 and/or SAA2, respectively, where a difference between the subject's levels and normal levels are indicative of obesity in the subject. As used herein, the term acute phase serum amyloid A protein (A-SAA) is the combination of SAA1 and SAA2. For example, an antibody that binds A-SAA is used to indicate that the antibody binds both SAA1 and SAA2 and does not distinguish between the two proteins. The terms “patient” and “subject” are used interchangeably herein. In particular, a subject may be a mammal, such as, but not limited to a mouse, rat, dog, horse or cat. More particularly, the subject is a human or non-human primate.

As used herein, the term “diagnose” means to confirm the results of other tests or to simply confirm suspicions that the subject may have an abnormal condition, such as obesity. A “test,” on the other hand, is used to indicate a screening method where the patient or the healthcare provider has no indication that the patient may, in fact, have an abnormal condition and may also be used to assess a patient's likelihood or probability of developing a disease or condition in the future. The methods of the present invention, therefore, may be used for diagnostic or screening purposes. Both diagnostic and testing can be used to “stage” the obese condition or obesity in a patient. As used herein, the term “stage” is used to indicate that the abnormal condition or obesity can be categorized, either arbitrarily or rationally, into distinct degrees of severity. The categorization may be based upon any quantitative characteristic that can be separated, such as, but not limited to, weight, BMI, blood pressure, a numerical value of a biomarker, e.g., A-SAA, insulin, or it may be based upon qualitiative characteristics that can be separated. The term “stage” may or may not involve disease progression.

As discussed, the invention relates to methods of diagnosing or testing for obesity in a subject, and “obesity,” as used herein, is used to mean that a subject is overweight or obese, or that a subject that has higher than normal levels of SAA1 and/or SAA2. In turn, an individual is overweight as assessed by Body Mass Index (BMI), which is a used to diagnose overweight and obesity. BMI is calculated by dividing an individual's weight in kilograms by the square of the individual's height in meters. Using BMI, the National Heart, Lung, and Blood Institute (NHLBI) has developed six weight categories, which are shown in Table 1 below. To be clear, “obesity” as used herein includes, but is not limited to, subjects that fall into the overweight and obese classes 1-3 weight categories as assessed by BMI. The obesity may be inflammatory obesity or non-inflammatory obesity.

TABLE 1

| Weight Classification | BMI (kg/m²) |
|---|---|
| Underweight | <18.5 |
| Normal | 18.5-24.9 |
| Overweight | 25.0-29.9 |
| Obese - Class 1 | 30.0-34.9 |
| Obese - Class 2 | 35.0-39.9 |
| Obese - Class 3 | ≧40.0 |

The diagnostic and screening methods comprise measuring levels of at least one biomarker selected from the group consisting of SAA1 and SAA2. In one specific embodiment of the present invention, at least SAA1 is measured. In another specific embodiment of the present invention, at least SAA2 is measured. In yet another specific embodiment, at least A-SAA (SAA1 and SAA2) is measured. Applicants discovered that SAA1 and SAA2 are markers for obesity and associated diseases or metabolic disorders, including, but not limited to, diabetes, hypertension, hyperlipidemia, hypercholesterolemia, inflammation and atherosclerosis. As used herein, an “abnormal condition” is used to mean a disease, a metabolic disorder or a condition in the subject that is normally not present in a healthy individual. In turn, a biomarker for an abnormal condition, as used herein, is used to indicate a compound, molecule, ion or other chemical entity whose detectable levels are correlated or can be correlated to a normal individual. In addition to an abnormal condition on the organism level, a biomarker may also be used to determine altered organ, tissue or cellular state. Examples of abnormal conditions include, but are not limited to, Inflammatory conditions such as Rheumatoid Arthritis (RA), primary biliary cirrhosis, chronic active hepatitis, infections (acute or chronic, sepsis), advanced cancer, diabetic nephropathy, Crohn's disease, ulcerative colitis, pancreatitis, asthma, allergic rhinitis.

“Normal levels” of a given biomarker may be assessed by measuring levels of the biomarker in a known healthy subject, including the same subject that is later screened or being diagnosed. Normal levels may also be assessed over a population sample, where a population sample is intended to mean either multiple samples from a single patient or at least one sample from a multiple of subjects. Normal levels of a biomarker, in terms of a population of samples, may or may not be categorized according to characteristics of the population including, but not limited to, sex, age, weight, BMI, ethnicity, geographic location, fasting state, state of pregnancy or post-pregnancy, menstrual cycle, general health of the patient, alcohol or drug consumption, caffeine or nicotine intake and circadian rhythms.

To diagnose or screen the subject, levels of SAA1 and/or SAA2 is measured in a given sample. As used herein, a sample can be any environment that may be suspected of containing the biomarker of interest. Thus, a sample includes, but is not limited to, a solution, a cell, a body fluid, a tissue or portion thereof, and an organ or portion thereof. Examples of animal cells include, but are not limited to mammalian cells such as, for example, bovine, equine, porcine, canine, feline, human and nonhuman primates. The scope of the invention should not be limited by the cell type assayed. Examples of samples or body fluids to be assayed include, but are not limited to, blood, plasma, serum, urine, saliva, milk, seminal plasma, synovial fluid, interstitial fluid, cerebrospinal fluid, lymphatic fluids, bile, amniotic fluid, adipose tissue and liver tissue. In one specific embodiment, the sample that is assayed is serum. The scope of the methods of the present invention should not be limited by the type of sample assayed.

The samples may or may not have been removed from their native environment. Thus, the portion of sample assayed need not be separated or removed from the rest of the sample or from a subject that may contain the sample. For example, the blood of a subject may be assayed for SAA1 and/or SAA2 without removing any of the blood from the patient. Of course, the sample may also be removed from its native environment. For example, the sample may be a tissue section that can be used in immunohistochemistry (IHC) techniques, and the antibodies of the present invention may be used in standard IHC techniques, where the antibodies are brought into contact with the sample and the binding of the antibody to the biomarker is detected using in standard immunohistochemistry techniques. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

The scope of the invention is not limited by methods of measuring levels of a given biomarker. For example, “levels” of SAA1 and/or SAA2 include, but are not limited to, protein levels, nucleic acids levels, e.g., mRNA or cDNA levels, receptor binding and protein activity. When protein is measured to assess SAA1 and/or SAA2 levels, the protein can be in tact protein, denatured protein or even partially digested. Protein levels may be assessed using standard techniques well known in the art such as, but not limited to, mass spectroscopy and ELISA, Western blotting and other immunoassays to name a few. The immunoassays which can be used include but are not limited to competitive and non-competitive assay systems using techniques such as radioimmunoassays, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and the like. Such assays are well-known in the art and are routine for assessing protein levels (see Ausubel et al, eds, 1994, Current Protocols in Molecular Biology, Vol. 1, John Wiley & Sons. Inc., New York, which is incorporated by reference).

In ELISAs the capture molecule that initially binds to the biomarker does not have to be conjugated to a label; instead, a labeled subsequent detection molecule (which may recognize the capture molecule) may be added to the well. One of skill in the art would be knowledgeable as to the parameters that can be modified to increase the signal detected as well as other variations of ELISAs known in the art. As used herein the term “capture molecule” is used mean a molecule, such as but not limited to, an antibody or receptor that immobilizes the biomarker by specifically binding to the biomarker. Further, a biomarker is “immobilized” if the biomarker or biomarker-capture molecule complex is separated or is capable of being separated from the remainder of the sample. When the capture molecule is coated to a well or other surface, a detection molecule may be added following the addition of the biomarker of interest to the wells. As used herein, a detection molecule is used to mean a molecule, such as an antibody or receptor, comprising a label. In a specific embodiment, the methods of the present invention comprise the use of a capturing antibody and a detection antibody to detect SAA1 or SAA2.

Detection of captured SAA1 and/or SAA2 protein, i.e., protein levels, may be accomplished by use of a labeled capture molecule or labeled detection molecule. A label, as used herein, is intended to mean a chemical compound or ion that possesses or comes to possess or is capable of generating a detectable signal. The labels of the present invention may be conjugated to the capture molecule or detection molecule, the biomarker of interest or a surface onto which the label and/or capture molecule or detection molecule is attached. Examples of labels includes, but are not limited to, radiolabels, such as, for example, ³H and ³²P, that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems. Additional examples of labels include, but are not limited to, a phosphorescent dye, a tandem dye and a particle. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label also includes a “tag” or hapten that can bind selectively to a conjugated molecule such that the conjugated molecule, when added subsequently along with a substrate, is used to generate a detectable signal. For example, one can use biotin as a label and subsequently use an avidin or streptavidin conjugate of horseradish peroxidate (HRP) to bind to the biotin label, and then use a colorimetric substrate (e.g., tetramethylbenzidine (TMB)) or a fluorogenic substrate such as Amplex Red reagent (Molecular Probes, Inc.) to detect the presence of HRP. Numerous labels are known by those of skill in the art and include, but are not limited to, particles, fluorophores, haptens, enzymes and their calorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9th edition, CD-ROM, (September 2002), which is herein incorporated by reference.

A fluorophore of the present invention is any chemical moiety that exhibits an absorption maximum beyond 280 nm, and when covalently attached to a labeling reagent retains its spectral properties. Fluorophores of the present invention include, without limitation; a pyrene (including any of the corresponding derivative compounds disclosed in U.S. Pat. No. 5,132,432, incorporated by reference), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Ser. Nos. 09/968,401 and 09/969,853, incorporated by reference), a carbocyanine (including any corresponding compounds in U.S. Ser. Nos. 09/557,275; 09/969,853 and 09/968,401; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1, incorporated by reference), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896, incorporated by reference), a xanthene (including any corresponding compounds disclosed in U.S. Pat. Nos. 6,162,931; 6,130,101; 6,229,055; 6,339,392; 5,451,343 and U.S. Ser. No. 09/922,333, incorporated by reference), an oxazine (including any corresponding compounds disclosed in U.S. Pat. No. 4,714,763, incorporated by reference) or a benzoxazine, a carbazine (including any corresponding compounds disclosed in U.S. Pat. No. 4,810,636, incorporated by reference), a phenalenone, a coumarin (including an corresponding compounds disclosed in U.S. Pat. Nos. 5,696,157; 5,459,276; 5,501,980 and 5,830,912, incorporated by reference), a benzofuran (including an corresponding compounds disclosed in U.S. Pat. Nos. 4,603,209 and 4,849,362, incorporated by reference) and benzphenalenone (including any corresponding compounds disclosed in U.S. Pat. No. 4,812,409, incorporated by reference) and derivatives thereof. As used herein, oxazines include resorufins (including any corresponding compounds disclosed in 5,242,805, incorporated by reference), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045, incorporated by reference), or a rhodamine (including any corresponding compounds in U.S. Pat. Nos. 5,798,276; 5,846,737; U.S. Ser. No. 09/129,015, incorporated by reference). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171, incorporated by reference). Alternatively, the fluorophore is a xanthene that is bound via a linkage that is a single covalent bond at the 9-position of the xanthene. Specific xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Specific fluorophores of the invention include xanthene (rhodol, rhodamine, fluorescein and derivatives thereof) coumarin, cyanine, pyrene, oxazine and borapolyazaindacene. More specific examples of fluorophores include, but are not limited to, sulfonated xanthenes, fluorinated xanthenes, sulfonated coumarins, fluorinated coumarins and sulfonated cyanines. The choice of the fluorophore attached to the labeling reagent will determine the absorption and fluorescence emission properties of the labeling reagent and immuno-labeled complex. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In one aspect of the invention, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp).

Many of fluorophores can also function as chromophores and thus the described fluorophores are also preferred chromophores of the present invention.

In addition to fluorophores, enzymes also find use as labels. Enzymes are desirable labels because amplification of the detectable signal can be obtained resulting in increased assay sensitivity. The enzyme itself may not produce a detectable signal but is capable of generating a signal by, for example, converting a substrate to produce a detectable signal, such as a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. The enzyme substrate is selected to yield the preferred measurable product, e.g. calorimetric, fluorescent or chemiluminescence. Such substrates are extensively used in the art, many of which are described in the MOLECULAR PROBES HANDBOOK.

In a specific embodiment, a colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other calorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042) and reduced dihydroxanthenes, including dihydrofluoresceins (U.S. Pat. No. 6,162,931, incorporated by reference) and dihydrorhodamines including dihydrorhodamine 123. Peroxidase substrates that are tyramides (U.S. Pat. Nos. 5,196,306; 5,583,001 and 5,731,158, incorporated by reference) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912, incorporated by reference) fluorescein diphosphate, 3-O-methylflubrescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986, incorporated by reference).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. Preferred fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236, incorporated by reference), 4-methylumbelliferyl beta-D-galactopyranoside, carboxyumbelliferyl beta-D-galactopyranoside and fluorinated coumarin beta-D-galactopyranosides (U.S. Pat. No. 5,830,912, incorporated by reference).

Additional enzymes include, but are not limited to, hydrolases such as cholinesterases and peptidases, oxidases such as glucose oxidase and cytochrome oxidases, and reductases for which suitable substrates are known.

Specific embodiments of the present invention comprise enzymes and their appropriate substrates to produce a chemiluminescent signal, such as, but not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

Additional embodiments comprise haptens such as biotin. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, effector molecules, growth factors, chemokines, cytokines, lymphokines, amino acids, peptides, chemical intermediates, nucleotides and the like.

Fluorescent proteins also find use as labels for the labeling reagents of the present invention. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorphore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556, incorporated by reference, and the sulforhodamine fluorophores disclosed in 5,798,276, or the sulfonated cyanine fluorophores disclosed in U.S. Ser. Nos. 09/968/401 and 09/969/853, incorporated by reference; or the sulfonated xanthene derivatives disclosed in 6,130,101, incorporated by reference and those combinations disclosed in U.S. Pat. No. 4,542,104, incorporated by reference. Alternatively, the fluorophore functions as the energy donor and the fluorescent protein is the energy acceptor.

In one embodiment, the label is a fluorophore selected from the group consisting of fluorescein, coumarins, rhodamines, 5-TMRIA (tetramethylrhodamine-5-iodoacetamide), (9-(2(or 4)-(N-(2-maleimdylethyl)-sulfonamidyl)-4(or 2)-sulfophenyl)-2,3,6,7,12,13,16,17-octahydro-(1H, 5H, 11H, 15H-xantheno(2,3,4-ij:5,6,7-i′j′)diquinolizin-18-ium salt) (Texas Red®), 2-(5-(1-(6-(N-(2-maleimdylethyl)-amino)-6-oxohexyl)-1,3-dihydro-3,3-dimethyl-5-sulfo-2H-indol-2-ylidene)-1,3-propyldienyl)-1-ethyl-3,3-dimethyl-5-sulfo-3H-indolium salt (Cy™3), N,N′-dimethyl-N-(iodoacetyl)-N′-(7-nitrobenz-2-oxa-1,3-diazol-4-yl)ethylenediamine (IANBD amide), 6-acryloyl-2-dimethylaminonaphthalene (acrylodan), pyrene, 6-amino-2,3-dihydro-2-(2-((iodoacetyl)amino)ethyl)-1,3-dioxo-1H-benz(de)isoquinoline-5,8-disulfonic acid salt (lucifer yellow), 2-(5-(1-(6-(N-(2-maleimdylethyl)-amino)-6-oxohexyl)-1,3-dihydro-3,3-dimethyl-5-sulfo-2H-indol-2-ylidene)-1,3-pentadienyl)-1-ethyl-3,3-dimethyl-5-sulfo-3H-indolium salt (Cy™5), 4-(5-(4-dimethylaminophenyl)oxazol-2-yl)phenyl-N-(2-bromoacetamidoethyl)sulfonamide (Dapoxyl® (2-bromoacetamidoethyl)sulfonamide)), (N-(4,4-difluoro-1,3,5,7-tetramethyl-4-bora-3a,4a-diaza-s-indacene-2-yl)iodoacetamide (BODIPY® 507/545 IA), N-(4,4-difluoro-5,7-diphenyl-4-bora-3a,4a-diaza-s-indacene-3-propionyl)-N′-iodoacetylethylenediamine (BODIPY 530/550 IA), 5-((((2-iodoacetyl)amino)ethyl) amino)naphthalene-1-sulfonic acid (1,5-IAEDANS), and carboxy-X-rhodamine, 5/6-iodoacetamide (XRIA 5,6). Another example of a label is BODIPY-FL-hydrazide. Other luminescent labels include lanthanides such as europium (Eu3+) and terbium (Tb3+), as well as metal-ligand complexes of ruthenium [Ru(II)], rhenium [Re(I)], or osmium [Os(II)], typically in complexes with diimine ligands such as phenanthroline.

Immunoprecipitation protocols generally comprise lysing a population of cells in a lysis buffer such as RIPA buffer (1% NP40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., Western blot analysis. One of skill in the art would be knowledgeable as to the parameters that can be modified to increase the binding of an antibody to the biomarker and decrease the background binding.

Western blot analysis generally comprises preparing protein samples, electrophoresis of the protein samples in a polyacrylamide gel (e.g., 8%-20% SDS-PAGE depending on the molecular weight of the biomarker), transferring the protein sample from the polyacrylamide gel to a membrane such as nitrocellulose, PVDF or nylon, blocking the membrane in blocking solution e.g., PBS with 3% BSA or non-fat milk), washing the membrane in washing buffer (e.g., PBS-Tween 20), blocking the membrane with primary antibody diluted in blocking buffer, washing the membrane in washing buffer, blocking the membrane with a secondary antibody (which recognizes the primary antibody, e.g., an anti-human antibody) conjugated to an enzymatic substrate, e.g., horseradish peroxidase or alkaline phosphatase or radioactive molecule (e.g., ³²P or ¹²⁵I diluted in blocking buffer, washing the membrane in wash buffer, and detecting the presence of the antigen. One of skill in the art would be knowledgeable as to the parameters that can be modified to increase the signal detected and to reduce the background noise.

The use of subsequent detection molecules to detect binding of the capture molecule to the biomarker may include, but are not limited to, radioactive isotopes and enzymes, such as horse radish peroxidase or alkaline phosphatase, as is described herein. Additionally, if the capture molecule, for example, is bound to a bead or particle, methods of detecting and measuring bound biomarker may also include flow cytometry (FACS).

Levels of SAA1 and/or SAA2 may also be assessed by measuring levels of related nucleic acids using standard techniques well-known in the art such as, but not limited to, PCR, RT-PCR, Northern blotting, Southern blotting and arrays. Such blotting and array techniques are well-known in the art and generally involve the nucleic acid of interest being hybridized to a complimentary nucleic acid that is labeled, or vice versa. Labels for nucleic acids include, but are not limited to, radioisotopes and other labels described herein.

Protein activity is used as it is in the art and indicates a direct or indirect downstream response to the addition or removal of the protein. Thus, protein activity includes but is not limited to, cellular or physiological responses caused by the biomarker or by the binding of the biomarker, e.g., SAA1 and/or SAA2 to its receptor and further downstream effects. It appears that the formyl peptide receptor-like 1/lipoxin A4 receptor (FPRL 1/LXA4R) is required for SAA-mediated IL-8 production in neutrophils (He R, et al., Blood 101:1572-1581 (2003), incorporated by reference). Furthermore, SAA activates nuclear factor kappa B (NFκB) and mitogen-activated protein (MAP) kinases ERK1/2 and p38, both of which are critical molecules in inflammation signaling pathway. In addition, A-SAA increases the activity of secretory phospholipase A2 (sPLA2). (See also Pruzanski W, et al., Biochem J. 1995 Jul. 15; 309 (Pt 2):461-4.) Accordingly, measuring SAA1 and/or SAA2 levels includes, but is not limited to, measuring levels or activities of enzymes, e.g., lipases, kinases, inflammatory cytokines, as well as levels of other effector molecules, e.g., activated nuclear transcription factors such as NFκB, that are known to be activated or deactivated by SAA1 and/or SAA2 or are known to be involved in the SAA1/SAA2 receptor pathway.

Once levels of SAA1 and/or SAA2 are measured, these measured levels are compared to normal levels of SAA1 and/or SAA2 to determine a difference, if any, between the measured levels and the normal levels of SAA1 and/or SAA2. A difference between normal levels and the measured levels of SAA1 and/or SAA2 may indicate that the subject is obese or has a higher (or lower) probability of becoming obese than normal subjects. In addition the magnitude of difference between measured levels and normal levels of SAA1 and/or SAA2 may also indicate the degree of obesity or the level of probability of becoming obese, compared to normal subjects.

Similarly, a difference between normal levels and the measured levels of SAA1 and/or SAA2 may indicate that the subject has a certain abnormal condition or has a higher (or lower) probability of developing an abnormal condition than in normal subjects. In addition the magnitude of difference between measured levels and normal levels of SAA1 and/or SAA2 may also indicate the degree of abnormality or the level of probability of developing the abnormal condition, compared to normal subjects.

The difference between measured levels of SAA1 and/or SAA2 and normal levels may be a relative or absolute quantity. Of course, the difference may be equal to zero, indicating that the patient is normal, or that there has been no change in levels of biomarker since the previous assay. The difference may simply be, for example, a measured fluorescent value, radiometric value, densitometric value, mass value etc., without any additional measurements or manipulations. Alternatively, the difference may be expressed as a percentage or ratio of the measured value of the antigen to a measured value of another compound including, but not limited to, a standard. The difference may be negative, indicating a decrease in the amount of measured biomarker over normal value or from a previous measurement, and the difference may be positive, indicating an increase in the amount of measured antigen over normal values or from a previous measurement. The difference may also be expressed as a difference or ratio of the antigen to itself, measured at a different point in time. The difference may also be determined using in an algorithm, wherein the raw data is manipulated.

In addition, A-SAA directly stimulates production of inflammatory cytokines in vascular endothelial cells and monocytes, extending a previous report that A-SAA induces interleukin 8 (IL-8) and TNFα production in human neutrophils and establishing SAA as a pro-inflammatory adipocytokine. Accordingly the present invention also provides diagnostic or screening methods comprising measuring levels of A-SAA and additional biomarkers that are also indicative of particular disease states. In one embodiment, for example, the present invention provides methods of diagnosing or screening a subject for obesity or associated abnormal conditions comprising measuring levels of SAA1 and/or SAA2 and also measuring levels of additional biomarkers including but not limited to IL-6, IL-8, MCP-1, PAI-1, TNFα and RANTES.

It is interesting to note that obesity is associated with low HDL of which SAA is a component. (Chan D C, et al., Am J Cardiovasc Drugs 4:227-246 (2004); Avenell A, et al., J Hum Nutr Diet 17:293-316 (2004); Wagh A, et al., Expert Rev Cardiovasc Ther 2:213-228 (2004), all of which are incorporated by reference) As the interaction of SAA with HDL may facilitate the latter's degradation, the invention teaches that the increase of adipose-derived A-SAA in obesity can be a mechanistic link between obesity, low HDL, and increased cardiovascular disease risk.

A-SAA stimulates the production of a number of cytokines from vascular endothelial cells and monocyte-derived cells, showing that A-SAA is a mediator of the inflammatory process. The inflammatory effect of A-SAA on vascular endothelial cells and monocytes confirms that A-SAA is a link between obesity and cardiovascular disease. In mice, increased atherosclerosis is associated with serum A-SAA level evoked by high-fat and high-cholesterol diet, which is independent of plasma lipoproteins, indicating that SAA may promote atherosclerosis directly (Lewis K E, et al., Circulation 110:540-545 (2004), incorporated by reference). In the Women's Ischemia Syndrome Evaluation (WISE) study, Johnson et al. found that A-SAA, compared to C-reactive protein (CRP), is independently associated with angiographic coronary artery disease and highly predictive of 3-year cardiovascular events (Johnson B D, et al., Circulation 109:726-732 (2004), incorporated by reference).

Accordingly, the present invention also provides methods of diagnosing or testing for an abnormal condition in a subject comprising measuring levels of serum amyloid A protein 1 (SAA1) and/or serum amyloid A protein 2 (SAA2) and comparing these measured levels with accepted normal levels of SAA1 and/or SAA2, respectively, where a difference between the subject's levels and normal levels are indicative of the presence of an abnormal condition in the subject.

The abnormal condition that is diagnosed or screened is often, but not necessarily, related to obesity in the subject. Thus the abnormal condition that is diagnosed or screened may be present in a subject that is, but not necessarily, obese. Examples of abnormal conditions that can be diagnosed or screened using the methods of the present invention include, but are not limited to diabetes, hypertension, dyslipidemia, hypercholesterolemia, inflammation and atherosclerosis. (See Haslam D W, et al. Lancet. 366(9492):1197-209 (2005) and Shaw D I, et al. Proc Nutr Soc. 64(3):349-57 (2005)). Other abnormal conditions include, but are not limited to, Inflammatory conditions such as Rheumatoid Arthritis (RA), primary biliary cirrhosis, chronic active hepatitis, infections (acute or chronic, sepsis), advanced cancer, diabetic nephropathy, Crohn's disease, ulcerative colitis, pancreatitis, asthma, allergic rhinitis. Still another abnormal condition that can be screened, diagnosed or treated is “metabolic syndrome,” which is a condition related to obesity. (See Dandona et al., Circulation, 111(11):1448-54 (2005), which is hereby incorporated by reference).

In one specific embodiment of diagnosing or screening for abnormal conditions, at least SAA1 is measured. In another specific embodiment of diagnosing or screening for abnormal conditions, at least SAA2 is measured. In yet another specific embodiment of diagnosing or screening for abnormal conditions, at least A-SAA (SAA1 and SAA2) is measured.

The present invention also provides methods of monitoring the progression of obesity or an abnormal condition in a subject, comprising measuring levels of serum amyloid A protein 1 (SAA1) and/or serum amyloid A protein 2 (SAA2) at two different time points in a subject, and comparing these measured levels at the two time points to determine a difference in levels of SAA1 and/or SAA2 over time, respectively, where a difference over time is indicative of the progression or regression of obesity or an abnormal condition in the patient. As used herein, the phrase “monitor the progression” is used to indicate that the abnormal condition in the subject is being periodically checked to determine if the abnormal condition is progression (worsening), regressing (improving) or remaining static (no detectable change) in the individual by assaying SAA1 and/or SAA2 levels in the subject using the methods of the present invention. The methods of monitoring may be used in conjunction with treatments for the abnormal condition to monitor the efficacy of the treatment. Thus, “monitor the progression” is also intended to indicate assessing the efficacy of a treatment regimen by periodically measuring levels of SAA1 and/or SAA2 and correlating the differences in SAA1 and/or SAA2 in the subject over time with the progression, regression or stasis of the abnormal condition. Monitoring may include two time points from which a sample is taken, or it may include more time points, where any of the levels of SAA1 and/or SAA2 at one particular time point from a given subject may be compared with SAA1 and/or SAA2 levels in the same subject, respectively, at one or more other time points.

The present invention also provides methods of treating obesity or an abnormal condition associated with obesity. As used herein, the term “treatment” is used to indicate a procedure which is designed ameliorate one or more causes, symptoms, or untoward effects of obesity or an abnormal condition in a subject. Likewise, the term “treat” is used to indicate performing a treatment. The treatment can, but need not, cure the subject, i.e., remove the cause(s), or remove entirely the symptom(s) and/or untoward effect(s) of the abnormal condition in the subject. Thus, a treatment may include treating a subject to attenuate symptoms such as, but not limited to, high blood pressure, fatigue, headaches, visual disturbances, nausea, vomiting, arterial disease and irregular heartbeat. The invention also provides methods of preventing or preventing the progression of abnormal conditions associated with obesity.

The present invention also relates to treating obesity and/or abnormal conditions comprising administering to a subject a compound that is capable of reducing the levels of active SAA1 and/or SAA2 in a patient in need thereof. “Active levels” as used in relation to SAA1 and/or SAA2 is intended to indicate quantities of SAA1 and/or SAA2 levels of activity of SAA1 and/or SAA2. Thus reducing active levels of SAA1 and/or SAA2 include but are not limited to, removing or reducing quantities of SAA1 and/or SAA2, or binding SAA1 and/or SAA2 in such a way that the molecule can not bind to a receptor or activate any biochemical pathways in the subject. Lowering active levels also includes binding the SAA 1 and/or SAA2 receptor with a compound to prevent SAA1 and/or SAA2 from binding thereto, respectively. In one embodiment, the methods comprise reducing the active levels of SAA1 in the subject. In another embodiment, the methods comprise reducing the active levels of SAA2 in a subject. In yet another embodiment, the methods comprise reducing the active levels of A-SAA in the subject. In one specific embodiment, the compound that is administered is a PPARγ agonist, such as, but not limited to, rosiglitazone. As used herein, the term “administer” and “administering” are used to mean introducing at least one compound into a subject. When administration is for the purpose of treatment, the substance is provided at, or after the onset of, a diagnosis of obesity or a diagnosis of the abnormal condition that is to be treated. The therapeutic administration of this substance serves to attenuate any symptom, or prevent additional symptoms from arising. When administration is for the purposes of preventing an abnormal condition from arising (“prophylactic administration”), the substance is provided in advance of any visible or detectable symptom or indication of the abnormal condition. The prophylactic administration of the substance serves to attenuate subsequently arising symptoms or prevent symptoms from arising altogether in a subject that may or may not be at risk. The route of administration of the composition includes, but is not limited to, topical, transdermal, intranasal, vaginal, rectal, oral, subcutaneous intravenous, intraarterial, intramuscular, intraosseous, intraperitoneal, epidural and intrathecal.

Furthermore, the methods of treating or preventing obesity or an abnormal condition invention also relate to coadministering one or more substances in addition to compounds that reduce levels of active SAA1 and/or SAA2. The term “coadminister” indicates that each of at least two compounds is administered during a time frame wherein the respective periods of biological activity or effects overlap. Thus the term includes sequential as well as coextensive administration of the compounds of the present invention. And similar to administering compounds, coadministration of more than one substance can be for therapeutic and/or prophylactic purposes. If more than one substance is coadministered, the routes of administration of the two or more substances need not be the same. The scope of the invention is not limited by the identity of the substance which may be coadministered. For example, rosiglitazone may be coadministered with another pharmaceutically active substance, such as but not limited to, sympatholytics or other a adrenergic agonists, α adrenergic receptor antagonists, β₁ and β₂ adrenergic antagonists (beta blockers), ACE inhibitors (angiotensin converting enzyme inhibitors), vasodilators, calcium channel blockers, HMG-CoA reductase inhibitors and insulin. Additional agents that may be co-administered include but are not limited to antibiotics.

The present invention also provides antibodies that are specific towards SAA1, SAA2 and/or A-SAA. Using the SAA1-specific peptide N′-AISDARENIQRFFG-C′ and the SAA2-specific peptide N′-VISNARENIQRLTG-3′ which are conjugated with KLH as antigens, the inventors have generated SAA1- and SAA2-specific polyclonal antibodies. The isoform-specific antibody detects ether SAA1 or SAA2, but not both proteins. As used herein, the term “antibody” is used to mean immunoglobulin molecules and functional fragments thereof, regardless of the source or method of producing the fragment. As used herein, a “functional fragment” of an immunoglobulin is a portion of the immunoglobulin molecule that specifically binds to a binding target. Thus, the term “antibody” as used herein encompasses whole antibodies, such as antibodies with isotypes that include but are not limited to IgG, IgM, IgA, IgD, IgE and IgY. Whole antibodies may be monoclonal or polyclonal, and they may be humanized or chimeric. The term “monoclonal antibody” as used herein is not limited to antibodies produced through hybridoma technology. Rather the term “monoclonal antibody” refers to an antibody that is derived from a single clone, including any eukaryotic, prokaryotic, or phage clone, and not the method by which it is produced. The term “antibody” also encompasses functional fragments of immunoglobulins, including but not limited to Fab fragments, Fab′ fragments, F(ab′)₂ fragments and Fd fragments. “Antibody” also encompasses fragments of immunoglobulins that comprise at least a portion of a V_{L} and/or V_{H} domain, such as single chain antibodies, a single-chain Fv (scFv), disulfide-linked Fvs and the like.

The antibodies of the present invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described in U.S. Pat. No. 5,939,598, which is herein incorporated by reference.

The antibodies of the present invention may be described or specified in terms of the epitope(s) or portion(s) of a polypeptide to which they recognize or specifically bind. Or the antibodies may be described based upon their ability to bind to specific conformations of the antigen, such as the conformation of the antigen, e.g., SAA1 or SAA2, when the antigen itself is bound to another molecule, such as the SAA receptor.

Antibodies of the present invention may also be described or specified in terms of their cross-reactivity, as well as their binding affinity towards the antigen. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The antibodies of the invention also include derivatives that are modified, for example, by covalent attachment of any type of molecule to the antibody such that covalent attachment does not prevent the antibody from generating an anti-idiotypic response. Examples of modifications to antibodies include but are not limited to, glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin and the like. Additionally, the derivative may contain one or more non-classical amino acids.

In one embodiment of the present invention, the antibodies are specific towards SAA1. In a more specific embodiment, antibodies with specificity towards SAA1 do not possess detectable binding affinity towards SAA2. Conversely, the invention also provides antibodies that are specific towards SAA2. In a more specific embodiment, antibodies with specificity towards SAA2 do not possess detectable binding affinity towards SAA1. Finally, the invention provides antibodies with specificity towards A-SAA.

The antibodies of the present invention may be generated by any suitable method known in the art. Polyclonal antibodies to SAA1 and/or SAA2 can be produced by various procedures well known in the art. For example, SAA1 and/or SAA2 can be administered to various host animals including, but not limited to, rabbits, mice, rats, to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (both of which are incorporated by reference).

Methods for producing and screening for specific antibodies using hybridoma technology are routine and well known in the art. In a non-limiting example, mice can be immunized with a fusion protein comprising SAA1 and/or SAA2. Once an immune response is detected, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones can then be assayed by methods known in the art for cells that secrete antibodies capable of binding a biomarker of the present invention. Ascites fluid, which generally contains high levels of antibodies, can be generated by immunizing mice with positive hybridoma clones.

The antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library. Phage expressing an antigen binding domain that binds the antigen of interest can be selected or identified with the antigen of interest, such as using a labeled antigen or antigen bound or captured to a solid surface or bead. The phage used in these methods are typically filamentous phage including, but not limited to, fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated by reference.

Antibody fragments which recognize specific epitopes, e.g., SAA1 and/or SAA2, may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab)₂ fragments). F(ab′)₂ fragments contain the variable region, the light chain constant region and the CH1 domain of the heavy chain.

Other methods, such as recombinant techniques, may be used to produce Fab, Fab′ and F(ab′)₂ fragments and are disclosed in PCT publication WO 92/22324; Mullinax et al., BioTechniques 12(6):864-869 (1992); and Sawai et al., AJRI 34:26-34 (1995); and Better et al., Science 240:1041-1043 (1988), which are herein incorporated by reference. After phage selection, for example, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

Examples of techniques which can be used to produce other types of fragments, such as scFvs and include those described in U.S. Pat. Nos. 4,946,778 and 5,258,498; Huston et al., Methods in Enzymology 203:46-88 (1991); Shu et al., Proc. Nat'l Acad. Sci. (USA) 90:7995-7999 (1993); and Skerra et al., Science 240:1038-1040 (1988), all of which are incorporated by reference. For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, all of which are herein incorporated by reference. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See U.S. Pat. No. 5,585,089; Riechmann et al., Nature 332:323 (1988), both of which are herein incorporated by reference. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Nat'l. Acad. Sci. 91:969-913 (1994)), and chain shuffling (U.S. Pat. No. 5,565,332), all of which are hereby incorporated by reference.

Completely human antibodies may be particularly desirable for therapeutic treatment or diagnosis of human patients. Human antibodies can be made by a variety of methods known in the art including phage display methods described above using antibody libraries derived from human immunoglobulin sequences. See also. U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated by reference.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, such as SAA1 and/or SAA2. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995), which is hereby incorporated by reference. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference.

Still another approach for generating human antibodies utilizes a technique referred to as guided selection. In guided selection, a selected non-human monoclonal antibody, e.g., a mouse antibody, is used to guide the selection of a completely human antibody recognizing the same epitope. (Jespers et al., Biotechnology 12:899-903 (1988), herein incorporated by reference).

The present invention also provides for kits for performing the methods described herein. Kits of the invention may comprise one or more containers containing one or more reagents useful in the practice of the present invention. Kits of the invention may comprise containers containing one or more buffers or buffer salts useful for practicing the methods of the invention. A kit of the invention may comprise a container containing a substrate for an enzyme. For example, a kit of the invention may comprise one or more substrates useful for detecting the enzymatic activity, i.e., horse radish peroxidase, or alkaline phosphatase.

Kits of the invention may comprise a container containing a stock antigen of known concentration. A stock of known concentration may be used to construct a calibration curve, for example. The calibration curve could then be used to determine the amount of antigen in a sample.

Kits of the invention may comprise one or more computer programs that may be used in practicing the methods of the invention. For example, a computer program may be provided that calculates a concentration of SAA1 and/or SAA2 in a sample from results of the detecting levels of antibody bound to the biomarker of interest. Such a computer program may be compatible with commercially available equipment, for example, with commercially available microplate readers. When determining the concentration of antigen in a sample, various dilutions of a stock of standard of known concentration may be applied to different wells in a microplate. Programs of the invention may take the output from microplate reader, prepare a calibration curve from the optical density observed in the wells and compare this densitometric reading to the optical density readings in wells with unknown amounts of antigen to determine how much antigen is present in the sample.

### EXAMPLES

Human Subjects. All human studies were approved by the Institutional Review Boards of the respective institutions and each participant provided written informed consent to participate. All blood samples were collected after overnight fasting and sera were stored at about −80° C. until used. All subjects were healthy according to medical history, physical examination, and laboratory testing unless otherwise specified in the protocols. None of the studied subjects showed clinical or laboratory evidence of acute inflammation.

Statistical Analysis. Results are expressed as mean±SEM. The Student's unpaired or paired t test was applied when appropriate as specified in the figure legends. Differences were considered to be significant at p<0.05. To control for relatedness among the Amish subjects, variance components analysis as implemented in SOLAR (Almasy L, et al., Am J Hum Genet 62:1198-1211 (1998), incorporated by reference) was used to assess the correlation between BMI and SAA levels in the larger set of 134 Amish individuals.

Methods

Study of body mass index (BMI) and serum SAA levels. Human subjects used in this study were a part of the Amish Family Diabetes Study, as described previously in Hsueh W C, et al., Diabetes Care 23:595-601 (2000) and Pollin T I, et al., Atherosclerosis 173:89-96 (2004), which are incorporated by reference. Initially, SAA levels were measured in plasma samples from 19 sex- and age-matched sets (within 5 years) of nondiabetic sibling pairs (38 individuals) with a discordance in BMI of at least 3 kg/m². These 38 individuals were then included in an expanded set of 134 nondiabetic individuals with BMI ranging from 17.0 to 41.8 kg/m². Blood samples were obtained from an antecubital vein after an overnight fast. BMI was calculated as weight (kg) divided by height (m) squared.

Weight loss study. 24 sedentary, overweight or obese (BMI 33±2 kg/m², mean±SEM), postmenopausal (57±1 yrs) women were studied before and after a 5-month dietary weight loss program. The intervention consisted of an outpatient hypocaloric feeding program (350 kcal/day deficit) in combination with aerobic exercise training. Fat mass was determined by dual-energy X-ray absorptiometry (Model DPX-L; Lunar Radiation, Madison, Wis.) using the 1.3z DPX-L extended analysis program. Fasting serum levels of SAA were measured at time 0 and after 5 months of weight loss program.

Rosiglitazone study. 8 nondiabetic healthy subjects (age 44.7±3.2 years, BMI 30.8±1.1 kg/m², mean±SEM) were recruited and treated with rosiglitazone (4 mg/day) for four weeks followed by 8 mg/day for eight weeks. Fasting blood was drawn every week during the intervention. Serum levels of SAA were measured at time 0 and after 12 weeks of rosiglitazone treatment. At the same time points, subcutaneous abdominal fat biopsies were obtained under local anesthesia for in vitro studies of SAA secretion, described elsewhere herein.

RNA Analysis. For microarray analysis, human omental and subcutaneous adipose were surgically obtained from 4 subjects with a BMI of 25, 28, 33 and 44 at the University of Maryland through an Institutional Research Board (IRB)-approved protocol. Isolated fat cells and stromal cells were obtained by collagenase digestion as described in Honnor R C, et al., J Biol Chem 260:15122-15129 (1985), incorporated by reference, in Kreb Ringer bicarbonate buffer containing about 4% albumin and about 200 nM adenosine (KRB-A). After centrifugation at about 200×g for 1-2 minutes, the media below the floating fat cells (containing the stromal-vascular cells (SVC) were removed and subjected to centrifugation at about 800×g for about 5 minutes. The pelleted cells were resuspended in KRB-A and washed 3 times using the same procedure. The floating fats cells were washed several times by floatation. RNA was extracted from fat cell and SVC fractions using the method of Chomzczynski and Sacchi as previously described in Fried S K, et al., J Clin Invest 92:2191-2198 (1993), incorporated by reference. For Northern analysis, human adipose tissue and liver specimens were purchased from the National Disease Research Interchange (Philadelphia, Pa.) and total RNAs were prepared with Trizol (Invitrogen, Carlsbad, Calif.) according to the manufacturer's instruction. All the other RNAs were purchased from Clontech (Palo Alto, Calif.).

RT-PCR Study. For semi-quantitative RT-PCR analysis, reverse transcription was carried out in a reaction containing about 1 μg of total RNA, polyT primer and MMLV reverse transcriptase using the Advantage kit (Clontech, Palo Alto, Calif.). PCR was performed under conditions typically consisting of about 28 cycles of about 94° C. for about 30 sec, about 55° C. for about 30 sec, and about 72° C. for about 1 minute. For detection of human A-SAA expression in fractionated adipose cells, primers 5′-GAGAGAAGCCAATTACATCGGC-3′ and 5′-AGTATITCTCAGGCAGGCCAGC-3′ were used. Human β-actin was amplified as a control with primers 5′-TTAATGTCACGCACGATITCC-3′ and 5′-AGACCTTCAACACCCCAGCCA-3′, see Xu H, et al., J Clin Invest 112:1821-1830 (2003), incorporated by reference. RT-PCR products were electrophoresed on about a 1% agarose gel, ethidium bromide stained, visualized by UV transillumination.

Northern Blotting. About fifteen micrograms of total RNA was loaded per lane for Northern analysis. A mouse multi-tissue blot was prepared from RNA of C57BL mice, see Yang R Z, et al., Biochem Biophys Res Commun 310:927-935 (2003), incorporated by reference. Human SAA2 cDNA corresponding to nucleotides 1 to 536 of BC020795 and murine SAA2 cDNA corresponding to nucleotides 1 to 565 of U60438 were used as probes for Northern analysis. These probes were about 97% (human) and about 95% (mouse) identical to SAA1 sequence and thus hybridized to both SAA1 and SAA2 under the hybridization and wash conditions used. The probes were random-labeled with ³²P-dCTP, and hybridization was carried out at about 65° C. in Rapid-hyb buffer (Amersham). Blots were washed twice with about 0.5×SSC/1% SDS at about 65° C. (stringent wash).

Secretion of SAA from adipose organ culture. Adipose organ culture followed the procedure previously described in Fried S K, et al, J Lipid Res 30:1917-1923 (1989), incorporated by reference. In a sterile hood, tissue was minced into about 5-10 mg pieces and washed with warm sterile saline. In the acute study of fat biopsies from humans treated with rosiglitazone, the adipose fragments were incubated for about 3 hours in M199-1% BSA (about 100 mg/ml) as previously described in Russell C D, et al., Am J Physiol 275:E507-515 (1998), incorporated by reference, and the culture media was collected and stored at about −80° C. until analysis. In the study of direct effect of rosiglitazone on adipose SAA production, adipose tissue fragments were cultured in M199-1% BSA (about 300-500 mg tissue/15 ml medium) with gentamicin (about 10 mg/L) at about 37° C. under an atmosphere of about 5% CO₂ in the absence of hormones, about 25 nM dexamethasone (American Pharmaceutical Partners, Schaumburg, Ill.) and about 7 mM insulin (Novo Nordisk, Princeton, N.J.) or a combination of these hormones with rosiglitazone (1 μM, GlaxoSmithKline, Philadelphia, Pa.). The culture media was changed daily and SAA was assayed in the conditioned medium of day 2.

Cell Culture Conditions. Primary human coronary artery endothelial cells (HCAECs) were purchased from Cambrex (Walkersville, Md.) and grown in endothelial cell basal medium-2 (EBM-2) supplemented with EGM-2 BulletKit. Experiments were conducted between the third to fifth passages. RAW264 cells (ATCC, Manassas, Va.) were grown in RPMI medium 1640 supplemented with about 10% fetal bovine serum. These cells were seeded on 6-well tissue culture plates at about 75% confluence and grown to about 90-95% confluence. The growth medium was replaced with supplement-free media (EBM-2 basal medium for HCAECs and RPMI1640 for RAW264). About one hour after the medium change, the cells were treated with recombinant human SAA (Peprotech, Rocky Hill, N.J.). The endotoxin level for this commercial preparation is advertised as less than about 0.1 ng/μg protein. The conditioned medium was collected about 8 hours after the SAA treatment by centrifugation at about 2,000×g for about 5 minutes and frozen until use for cytokine analysis.

Cytokine analysis. Human SAA (BioSource, Camarillo, Calif.) and PAI-1 (American Diagnostica, Greenwich, Conn.) were measured in duplicate with enzyme-linked immunosorbent assay (ELISA) kits according to instructions of the manufacturers. The SAA ELISA kit detects only A-SAA (SAA1 and SAA2) but not SAA4. The intra- and inter-assay CV is about 5% and about 8% respectively. Human MCP-1, IL-6, and IL-8, and mouse TNFα, IL-6, RANTES and MCP1 were analyzed at the Cytokine Core Facility, University of Maryland with cytokine multiplex reagents (Upstate Biotechnology, Inc., Lake Placid, N.Y.) by Luminex 100 (Luminex Corporation, Austin, Tex.). All samples were assayed in duplicate.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

Results

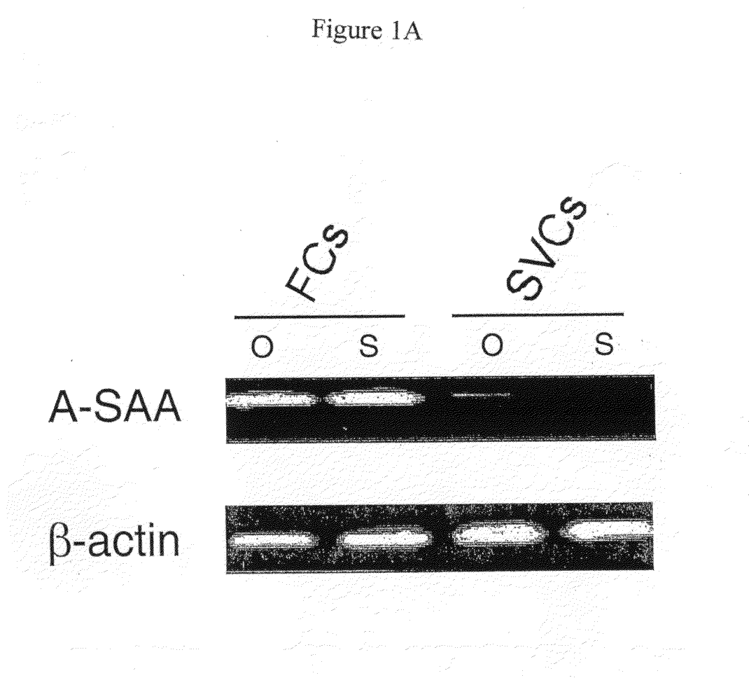

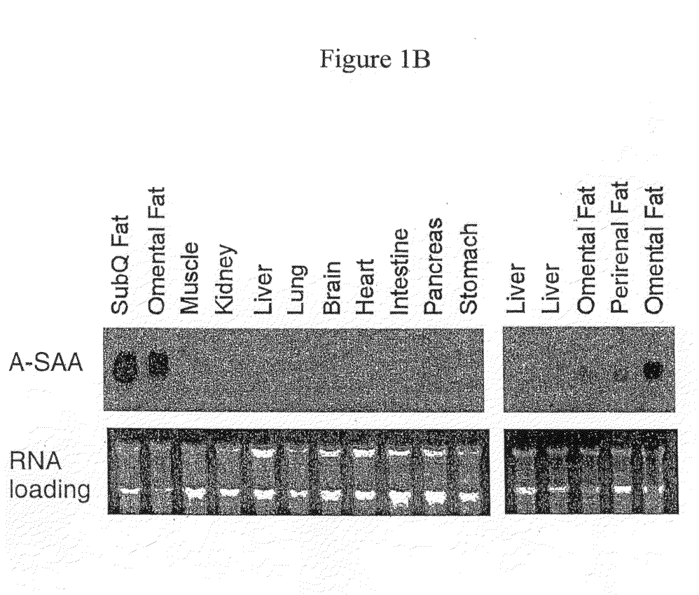

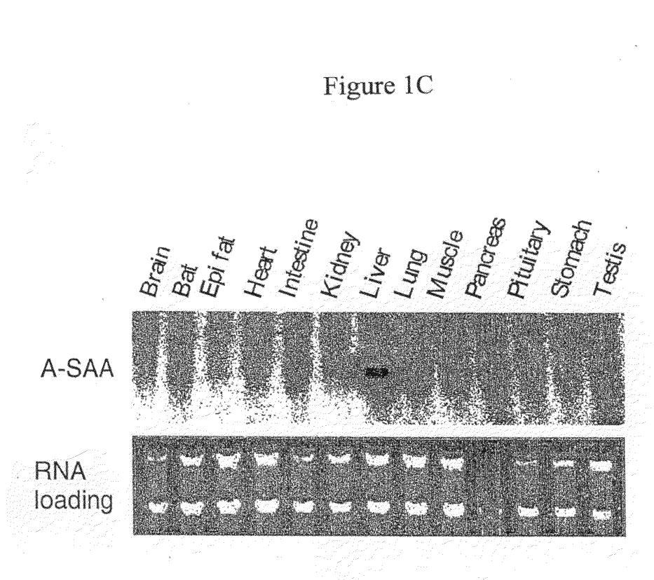

Acute-phase SAA is selectively expressed in human adipose tissue. To identify genes preferentially expressed in adipocytes, human adipose tissue was fractionated into adipocyte and stromal vascular cell (SVC) fractions and a microarray analysis (Affymetrix) was performed on the human U133A gene chip that contains about 29,000 gene transcripts. Preliminary data analysis indicated that about 800 genes were preferentially expressed in adipocytes (>3 fold) compared to SVC. Of these, acute-phase SAA (A-SAA) was one of the ten most abundant genes in adipocytes. RT-PCR analysis validated the high expression of A-SAA in human adipocytes, but not in SVCs and there was no significant difference in expression between subcutaneous and omental fat depots (FIG. 1A). Since human SAA1 and SAA2 are about 97% identical at the cDNA level, SAA2 was used as a probe for A-SAA (SAA1 and SAA2) gene expression by Northern analysis. FIG. 1B (top, left panel) shows that A-SAA is selectively and abundantly expressed in human adipose tissue but much less in the liver. The higher expression of A-SAA in adipose than in liver tissue (about 15-fold) was confirmed in an independent Northern blot containing additional specimens of adipose and liver tissues (FIG. 1B, top, right panel). Conversely in mice, A-SAA is predominately expressed in liver but not in adipose tissue as Northern blot analysis demonstrated using a mouse SAA2 cDNA probe, which is about 95% identical to mouse SAA1 cDNA (FIG. 1C). Furthermore, using an isoform-specific PCR primer, RT-PCR analysis revealed that SAA1 and SAA2 are approximately equally expressed in human adipose tissues. These studies demonstrate that in humans, A-SAA is predominately expressed in adipose tissue, more specifically in adipocytes.

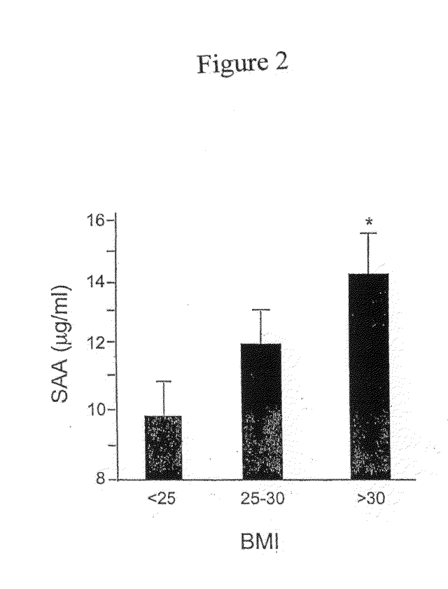

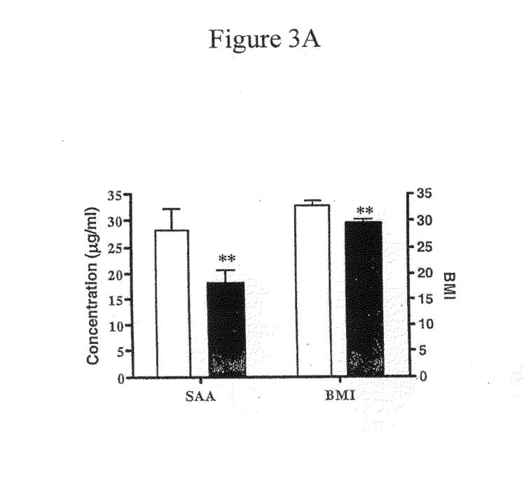

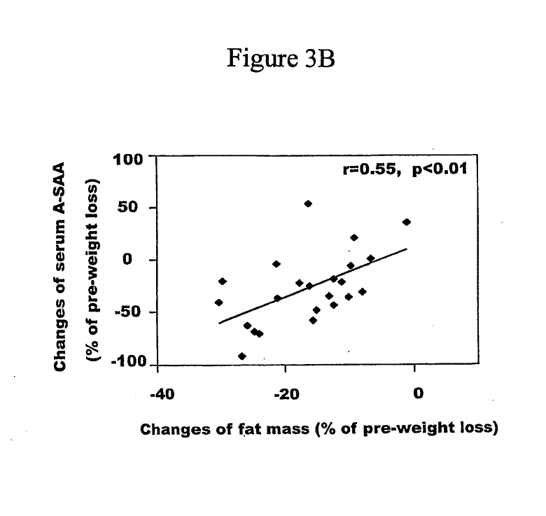

Serum A-SAA is increased with body mass index (BMI) and reduced after weight loss in humans. The direct correlation in serum A-SAA levels and body fat mass was confirmed by measuring plasma A-SAA levels in 19 age- and sex-matched nondiabetic sibling pairs who were discordant (>3 kg/m²) for BMI. A paired t-test showed significantly higher A-SAA levels in the obese siblings (p=0.044) and a positive Spearman correlation between the BMI and SAA differences (r=0.54, p=0.017). In an expanded set of 134 nondiabetic men and women over a range of BMIs, BMI was a significant predictor of ln-transformed SAA levels (p=0.025, controlling for age, sex and family structure). When subjects were grouped (FIG. 2) into lean (BMI<25 kg/m²), overweight (25 kg/m²<BMI<30 kg/m²) and obese (BMI≧30 kg/m²), the serum SAA level of the obese group (ln-transformed for analysis, back-transformed for presentation) was about 43% higher than that of the lean group (p=0.013, adjusted for age, sex and family structure). In a separate group of 24 women who were subjected to 8.7±5.1 kg (−10±2%) weight loss with a BMI decrease of about 4 kg/m² (33.0±4.1 kg/m² to 29.0±3.8 kg/m² (mean±S.D.)) through hypocaloric diet and exercise, a marked reduction in serum A-SAA was observed in 19 out of the 24 women, with an average about 27% reduction of SAA (FIG. 3A, p<0.01). Moreover, the changes in serum SAA concentration was correlated with changes of body fat mass (FIG. 3B, r=0.55, p<0.01), and was not correlated with changes of fat free body mass (r=0.23, p=0.3).

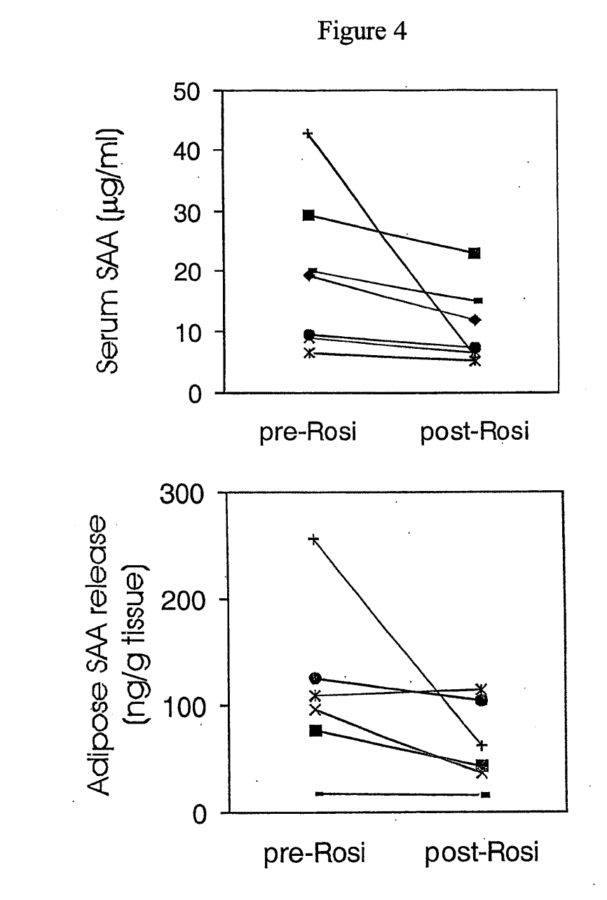

Rosiglitazone reduces serum SAA and suppresses adipose SAA production. The PPARγ agonist, rosiglitazone, was able to regulate A-SAA. While there were no statistically significant changes of body weight or fat mass in the subjects during the 12-week intervention, serum A-SAA levels were greatly reduced after the treatment in all the subjects (p<0.05) (FIG. 4). In addition, adipose tissue secretion of A-SAA was significantly reduced from these same subjects receiving rosiglitazone (FIG. 4). Accordingly, the invention provides methods of lowering serum A-SAA levels comprising administering rosiglitazone to a subject in need thereof.

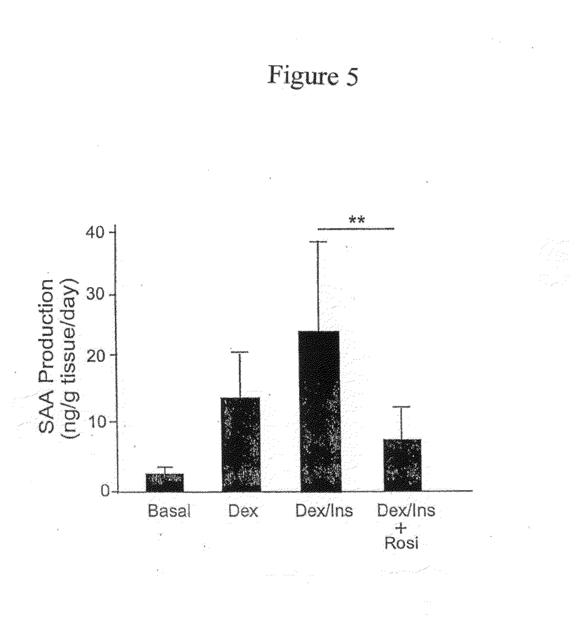

Rosiglitazone was shown to inhibit adipose A-SAA secretion by direct interaction with adipose tissue. Adipose biopsies were obtained from non-rosiglitazone-treated human subjects and cultured ex vivo, and a low basal level of A-SAA was detected in the culture media. Culturing the tissue in media containing dexamethasone (25 nM) and insulin (7 nM) resulted in higher A-SAA production as compared to culturing without hormones (FIG. 5). Addition of rosiglitazone, however, reduced this insulin/dexamethasone stimulation of A-SAA secretion in the media A-SAA by about 70%.

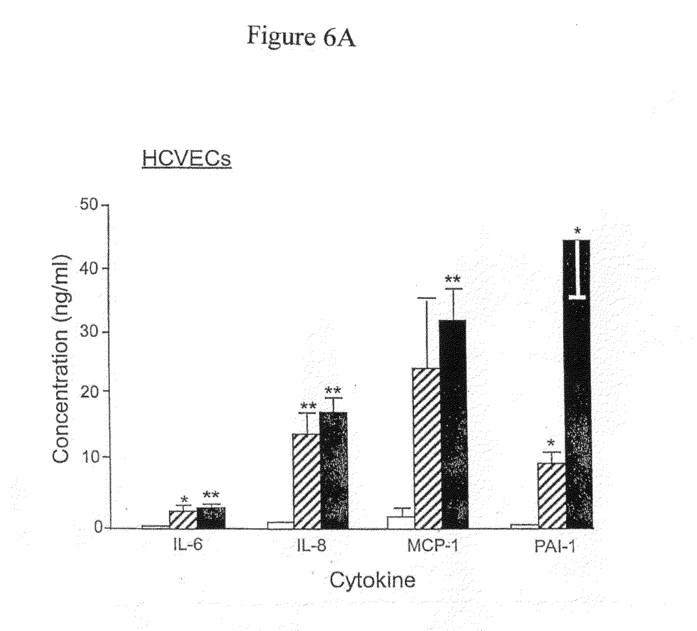

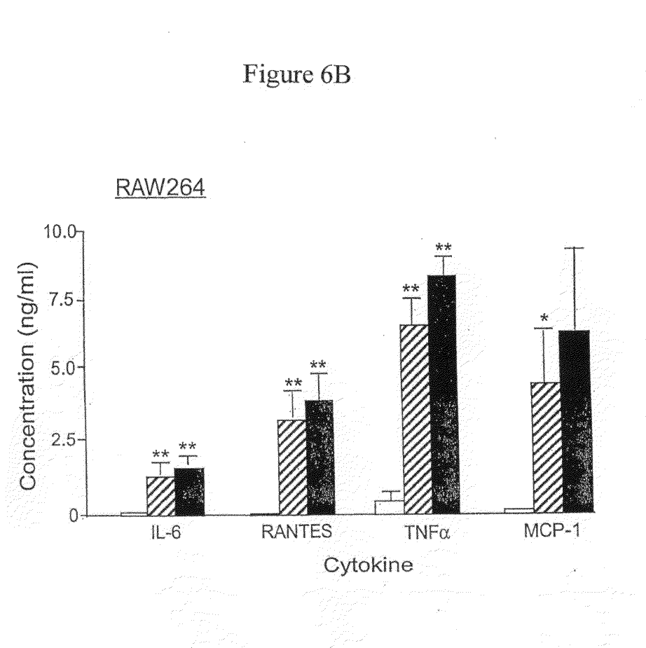

SAA is a pro-inflammatory cytokine. The results of the methods described herein provide a link between obesity and inflammation, and confirm that A-SAA is pro-inflammatory mediator that is involved in inflammation associated with obesity. Primary human coronary vascular endothelial cells (HCVECs) and the mouse monocyte cell line RAW264 were treated with vehicle (PBS), and low (0.47 μg/ml) or high (2.3 μg/ml) concentration of SAA for about 8 hours and the conditioned media was assayed for cytokine production. As shown in FIG. 6A, addition of A-SAA to culture media dramatically stimulated the release of IL-6, IL-8, MCP-1, and plasminogen activator inhibitor-1 (PAI-1) in HCVECs, and IL-6, IL-8, TNFα and RANTE in RAW264 cells, in a dose-dependent manner. To test for contamination of the human recombinant A-SAA with LPS, 1 ng/ml of LPS, which is a concentration that is about 15 times higher than the maximum possible contamination of endotoxin in the recombinant A-SAA, did not stimulate cytokine secretion in either of the cell cultures.

What is claimed is:

1. A method of diagnosing obesity, said method comprising
a) measuring levels of at least one biomarker selected from the group consisting of serum amyloid A1 (SAA1) and serum amyloid A2 (SAA2) in a sample;
b) comparing said measured levels of said at least one biomarker with normal levels of said at least one biomarker; and
c) determining a difference between said measured levels of said at least one biomarker and said normal levels of said at least one biomarker;
wherein a difference between said measured levels of said at least one biomarker and said normal levels of said biomarker, is indicative of obesity.

2. The method of claim 1, wherein said at least one biomarker is SAA1.

3. The method of claim 1, wherein said at least one biomarker is SAA2.

4. The method of claim 1, wherein both SAA1 and SAA2 are measured.

5. The method of claim 4, wherein said sample is selected from the group consisting of whole blood, plasma, serum, omental adipose tissue, subcutaneous adipose tissue and liver tissue.

6. The method of claim 5, wherein said sample is serum.

7. The method of claim 1, wherein said measuring said SAA1 and SAA2 comprises measuring a nucleic acid.

8. The method of claim 1, wherein said measuring said SAA1 and SAA2 comprises measuring a polypeptide.

9. The method of claim 1, wherein said measuring said SAA1 and SAA2 comprises assaying protein activity.

10. The method of claim 1, wherein said obesity is either inflammatory or non-inflammatory obesity.

11. A method of diagnosing an abnormal condition, said method comprising
a) measuring levels of at least one biomarker selected from the group consisting of serum amyloid A1 (SAA1) and serum amyloid A2 (SAA2) from said subject;
b) comparing said measured levels of said at least one biomarker with normal levels of said at least one biomarker; and
c) determining a difference between said measured levels of said at least one biomarker and said normal levels of said at least one biomarker;
wherein a difference between said measured levels of said at least one biomarker and said normal levels of said biomarker, is indicative of the presence of an abnormal condition.

12. The method of claim 11, wherein said abnormal condition is selected from the group consisting of diabetes, hypertension, dyslipidemia, atherosclerosis, hypercholesterolemia inflammation and infection.

13. The method of claim 11, wherein said at least one biomarker is SAA1.

14. The method of claim 11, wherein said at least one biomarker is SAA2.

15. The method of claim 11, wherein both SAA1 and SAA2 are measured.

16. The method of claim 11, wherein said sample is selected from the group consisting of whole blood, plasma, serum, omental adipose tissue, subcutaneous adipose tissue and liver tissue.

17. The method of claim 16, wherein said sample is serum.

18. The method of claim 11, wherein said measuring said SAA1 and SAA2 comprises measuring a nucleic acid.

19. The method of claim 11, wherein said measuring said SAA1 and SAA2 comprises measuring a polypeptide.

20. The method of claim 11, wherein said measuring said SAA1 and SAA2 comprises assaying protein activity.

21. A method of monitoring the progression of a disease state, said method comprising,
a) measuring levels of at least one biomarker selected from the group consisting of serum amyloid A1 (SAA1) and serum amyloid A2 (SAA2) at a first and second time point;
b) comparing said measured levels of said at least one biomarker at said first time point with measured levels of said at least one biomarker at said second time point; and
c) determining a difference between said measured levels of said at least one biomarker at said first and second time points;
wherein a difference between said measured levels of said at least one biomarker at said first and second time points indicates the progression of said abnormal condition.

22. The method of claim 21, wherein said at least one biomarker is SAA1.

23. The method of claim 21, wherein said at least one biomarker is SAA2.

24. The method of claim 21, wherein both SAA 1 and SAA2 are measured.

25. The method of claim 21, wherein said sample is selected from the group consisting of whole blood, plasma, serum, omental adipose tissue, subcutaneous adipose tissue and liver tissue.

26. The method of claim 25, wherein said sample is serum.

27. The method of claim 21, wherein said measuring said SAA1 and SAA2 comprises measuring a nucleic acid.

28. The method of claim 21, wherein said measuring said SAA1 and SAA2 comprises measuring a polypeptide.

29. The method of claim 21, wherein said measuring said SAA1 and SAA2 comprises assaying protein activity.

30. A method of treating obesity in a patient in need of treatment thereof, said method comprising reducing active levels in said patient of at least one polypeptide selected from the group consisting of serum amyloid A protein 1 (SAA1) and serum amyloid A protein 2 (SAA2).

31. The method of claim 30, wherein said at least one polypeptide is SAA1.

32. The method of claim 30, wherein said at least one polypeptide is SAA2.

33. The method of claim 30, wherein the active levels of both SAA1 and SAA2 are reduced.

34. A method of treating an abnormal condition in a patient in need of treatment thereof, said method comprising reducing an active level in said patient of at least one polypeptide selected from the group consisting of serum amyloid A protein 1 (SAA1) and serum amyloid A protein 2 (SAA2).

35. The method of claim 34, wherein said at least one polypeptide is SAA1.

36. The method of claim 34, wherein said at least one polypeptide is SAA2.

37. The method of claim 34, wherein the active levels of both SAA1 and SAA2 are reduced.

38. The method of claim 34, wherein said abnormal condition is selected from the group consisting of diabetes, hypertension, dyslipidemia, atherosclerosis and hypercholesterolemia.

39. A method of assessing the efficacy of a treatment regimen for treating an abnormal condition in a subject being treated for said abnormal condition, said method comprising,
a) measuring levels of at least one biomarker selected from the group consisting of serum amyloid A1 (SAA1) and serum amyloid A2 (SAA2) at a first and second time point;
b) comparing said measured levels of said at least one biomarker at said first time point with measured levels of said at least one biomarker at said second time point; and
c) determining a difference between said measured levels of said at least one biomarker at said first and second time points;
wherein a difference between said measured levels of said at least one biomarker at said first and second time points indicates the effectiveness of said treatment.

40. The method of claim 39, wherein said abnormal condition is selected from the group consisting of diabetes, hypertension, dyslipidemia, atherosclerosis and hypercholesterolemia.

41. A method of staging obesity, said method comprising
a) measuring levels of at least one biomarker selected from the group consisting of serum amyloid A1 (SAA1) and serum amyloid A2 (SAA2) in a sample; and
b) determining where said measured levels of said at least one biomarker fall within predetermined staged levels of said at least one biomarker;
wherein said predetermined staged levels of said biomarker are used to categorize the stage of obesity.

42. The method of claim 41, wherein said stage of obesity is either inflammatory obesity or non-inflammatory obesity.
